(19) United States
(12) Patent Application Publication
(10) Pub. No.: US 2015/0112400 A1
(43) Pub. Date: Apr. 23, 2015

(54) EXTERNAL DEFIBRILLATOR, EXPANDER THEREOF, AND DEFIBRILLATION GUARDING SYSTEM

(71) Applicant: SHENZHEN MINDRAY BIO-MEDICAL ELECTRONICS CO., LTD, Shenzhen, Guangdong (CN)

(72) Inventor: Min An, Shenzhen (CN)

(21) Appl. No.: 14/343,304

(22) PCT Filed: Sep. 3, 2012
(86) PCT No.: PCT/CN2012/080927

§ 371 Date: Nov. 13, 2014

(30) Foreign Application Priority Data
Sep. 6, 2011 (CN) 201110262754.0

(51) Int. Cl. A61N 1/39

(52) U.S. Cl. CPC A61N 1/3993; A61N 1/3975; A61N 1/3925
USPC 607/5

(57) ABSTRACT

This disclosure relates to external defibrillators, expanders thereof and defibrillation monitoring systems. The external defibrillator can include a first human-computer interaction unit, a main control unit, a power module, and a first communication interface for transmitting data with an expander corresponding to the external defibrillator. The expander can include a host for collecting vital sign data, and a second communication interface, connected with the host, for transmitting data with the external defibrillator. The external defibrillator is portable for emergency rescue and also transmits data with the expander, thereby realizing various expanded functions.

### TECHNICAL FIELD

This disclosure relates generally to medical devices. Particularly, this disclosure relates to external defibrillators, expanders thereof and defibrillation monitoring systems.

### SUMMARY

Disclosed herein are embodiments of external defibrillators, expanders thereof and defibrillation monitoring systems.

In one aspect, an external defibrillator can include a first human-computer interaction unit, a main control unit, a power module, and

a first communication interface for transmitting data with an expander corresponding to the external defibrillator.

In another aspect, an external defibrillator expander can include

a host for collecting vital sign data, and a second communication interface, connected with the host, for transmitting data with an external defibrillator.

In still another aspect, an defibrillation monitoring system can include an external defibrillator and an expander corresponding to the external defibrillator.

The external defibrillator can include a first human-computer interaction unit, a main control unit, a power module and a first communication interface for transmitting data with the expander. The expander can include a host for collecting vital sign data, and a second communication interface, connected with the host, for transmitting data with the external defibrillator.

### BRIEF DESCRIPTION OF THE DRAWINGS

FIG. 1 is a front view of an external defibrillator according to an embodiment;

FIG. 2 is a rear view of the external defibrillator in FIG. 1.

FIG. 3 is a perspective view of an expander according to an embodiment.

FIG. 4 shows a schematic diagram of a defibrillation monitoring system according to an embodiment.

### DETAILED DESCRIPTION

AED (Automated External Defibrillator) is a device for treating cardiac arrest. Conventional AED is portable to rescue sites due to its light weight and small size, but may only provide certain emergency functions, such as defibrillation, ECG data collection, and the like, and cannot communicate with a remote device. So conventional AEDs may not fully meet the needs of medical professionals. A defibrillation monitor has more functions than AED: it can provide not only defibrillation but also patient monitoring on vital signs, such as ECG, NIBP, EtCO2, IBP, etc. But, due to its large size and heavy weight, the traditional defibrillation monitor is often not portable for emergency rescue.

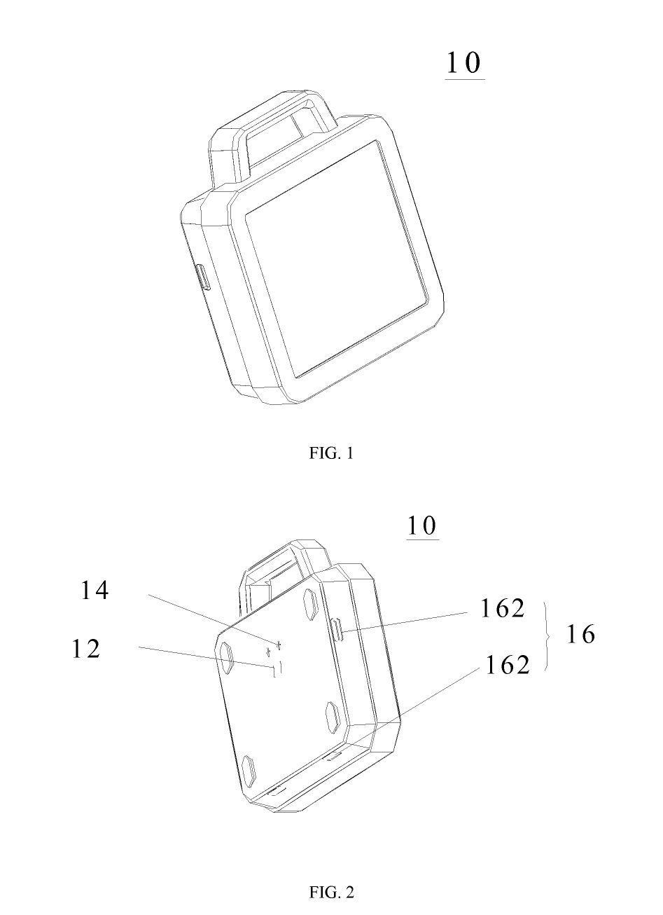

As shown in FIGS. 1 and 2, in one embodiment, an external defibrillator 10 may include a first human-computer interaction unit, a main control unit, a power module and a first communication interface 12, where the first communication interface 12 can be used for transmitting data between the external defibrillator 10 and its corresponding expander.

In one embodiment, the first human-computer interaction unit may include a display device (e.g., liquid crystal display (LCD)), a power switch, an input device (e.g., touch screen, key board), etc, and can be used for acquiring control commands inputted by a user. Through the first human-computer interaction unit, the user can start the external defibrillator 10, input control commands, set parameters, etc. The main control unit may include a processor, a memory and the like, and can be used for collecting at least 1-lead ECG data (for example, 3-, 5- or 12-lead ECG data) and/or defibrillation data according to the control command inputted by the user or an internal trigger command, and for processing the collected data. The memory of the main control unit can also store the collected data. In addition, the data collected by the main control unit may be displayed by the human-computer interaction unit.

In one embodiment, data transmitted by the first communication interface 12 may include one or more of the following data: vital sign data outputted by the expander corresponding to the external defibrillator, control and trigger commands outputted by the main control unit. The first communication interface 12 can transmit the control and trigger commands outputted by the main control unit to the expander so as to allow the expander to operate according to the control or trigger commands. For example, the main control unit can transmit the control or trigger command, such as start and shutdown commands, to the expander through the first communication interface 12.

In one embodiment, data transmitted by the first communication interface 12 may also include at least 1-lead ECG data and/or defibrillation data collected by the main control unit. In addition, in this embodiment or in another embodiment, the first human-computer interaction unit of the external defibrillator 10 can also be used for displaying vital signs outputted by the expander. Moreover, the at least 1-lead ECG data and/or defibrillation data collected by the main control unit may be transmitted to the expander through the first communication interface 12, and then processed, stored and outputted by the expander.

In some embodiments, the first communication interface 12 can include a first cable communication interface or a first wireless communication interface or both. The first wireless communication interface may be one or more of the following communication protocols: infrared, Bluetooth, WiFi, Zigbee and radio frequency (RF).

In addition, in one embodiment, the first wireless communication interface can communicate with a remote terminal and/or a remote server. The external defibrillator 10 can transmit the at least 1-lead ECG data and/or defibrillation data collected by the main control unit to the remote terminal and/or server through the first wireless communication interface, and then the remote terminal and/or server can process, store, display, print such data. Moreover, in case of malfunction, the external defibrillator 10 can also send warning to the remote terminal or server in terms of message, telephone, email etc. Thus, onsite rescue information or request can be sent to medical or relevant staff via message, telephone, email etc.

In one embodiment, as shown in FIG. 2, the external defibrillator 10 can further include a charging interface 14 for charging the power module. The charging interface 14 may be a cable charging interface or a wireless charging interface.

In one embodiment, as shown in FIG. 2, the external defibrillator 10 can further include a first locking device 16 for affixing the external defibrillator 10 to the expander. The first locking device 16 may include at least one first recess 162. In some embodiments, two first recesses 162 can be disposed respectively at side and bottom of the external defibrillator 10.

In one embodiment, the external defibrillator 10 can be an AED. In this disclosure, an AED can be a fully-automatic AED or a semi-automatic AED.

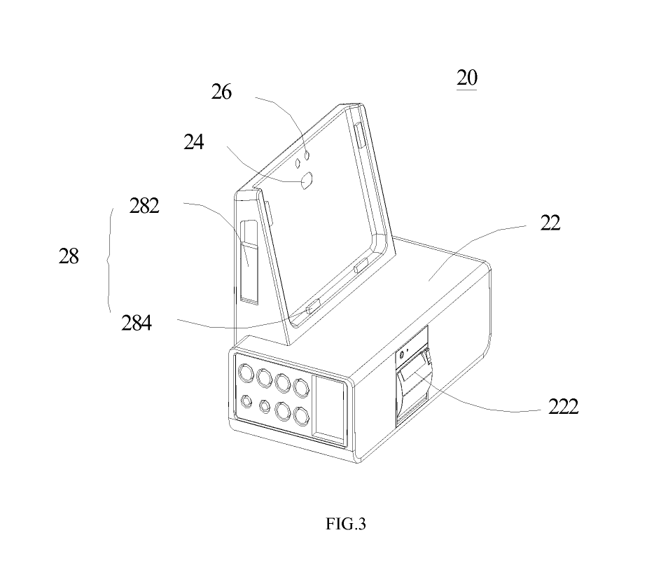

As shown in FIG. 3, in one embodiment, an external defibrillator expander 20 may include a host 22 and a second communication interface 24, where the host 22 can collect vital sign data and the second communication interface 24 can be connected with the host 22 to transmit data with the external defibrillator 10.

In some embodiments, the host 22 may include a vital sign data collection unit for collecting vital sign data, such as SpO2 (oxygen saturation), NIBP (non-invasive blood pressure), EtCO2 (end-tidal CO2 tension), IBP (invasive blood pressure), TEMP (temperature), RESP (respiration) etc. In some embodiments, the vital sign data collection unit can be one or more existing modules selected from the following modules: SpO2 measurement module, NIBP measurement module, EtCO2 measurement module.

In one embodiment, data transmitted by the second communication interface 24 may include one or more of the following data: vital sign data outputted by the expander 20, control and trigger commands outputted by the external defibrillator 10. When the vital sign data outputted by the expander 20 is transmitted to the external defibrillator 10 via the second communication interface 24, the data can be displayed by the first human-computer interaction unit of external defibrillator 10. In addition, after the expander 20 receives the control and/or trigger commands outputted by the external defibrillator 10, the expander 20 can operate according to the received commands.

In one embodiment, data transmitted by the second communication interface 24 can further include ECG data and/or defibrillation data; the host 22 can further include a recorder 222 that may be used for printing one or more of the ECG data and defibrillation data received by the second communication interface 24 and the vital sign data collected by the host 22. The second communication interface 24 may receive at least 1-lead ECG data and/or defibrillation data collected by the external defibrillator 10 and then print the received data using the recorder 222. In one embodiment, the recorder 222 can have a specification of 50 mm, 80 mm, 114 mm, 210 mm or the like. Since the recorder 222 may be compatible with a variety of specifications, it can meet different user needs, including better printout of 12-lead ECG report.

In some embodiments, the second communication interface 24 may include a second cable communication interface or a second wireless communication interface or both. The second wireless communication interface can be one or more of the following communication protocols: infrared, Bluetooth, WiFi, Zigbee and RF.

In one embodiment, as shown in FIG. 3, the expander 20 may further include a charging unit 26 for charging the external defibrillator 10 that corresponds to the charging interface 14 of the external defibrillator 10. The expander 20 can further include an AC or DC power source for charging the expander 20. The charging unit 26 may include AC/DC or DC/DC conversion circuitry for converting an external voltage to a voltage needed by the external defibrillator 10. In some embodiments, the charging unit 26 can charge the power module of the external defibrillator 10 using a cable charging interface or a wireless charging interface.

In another embodiment, the expander 20 may further include a second locking device 28 for affixing the external defibrillator 10 on the expander 20. As shown in FIG. 3, the second locking device 28 can include a locking wrench 282 and at least one second recess 284 corresponding to the first recess 162 on the external defibrillator 10. The locking wrench 282 can be disposed on the side of the expander 20. The external defibrillator 10 can be affixed on the expander 20 by pulling the locking wrench 282 so as to lock the second recess 284 and the first recess 162 together.

In another embodiment, the expander 20 can further include a second human-computer interaction unit that may include an input device and a display device. In this embodiment, the operation of the expander 20 can be controlled through the input device, and the vital sign data collected by the host 22 and the ECG data and/or defibrillation data received by the second communication interface 24 can be displayed by the display device.

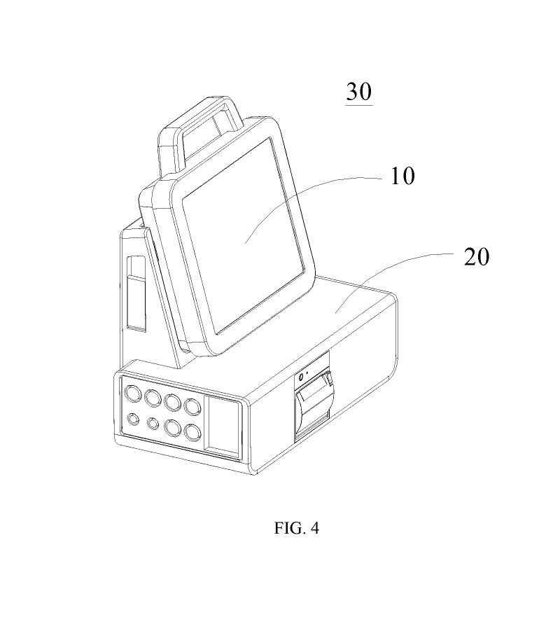

As shown in FIG. 4, in one embodiment, a defibrillation monitoring system 30 may include the external defibrillator 10 and the expander 20 described above. The external defibrillator 10 can transmit data with the expander 20 through the first communication interface 12, and the expander 20 can transmit data with the external defibrillator 10 through the second communication interface 24.

In one embodiment, the defibrillation monitoring system 30 can operate as follows:

When the system 30 is inactive, as shown in FIG. 4, the external defibrillator 10 is affixed on the expander 20. If the system 30 is needed to be used, the power switch of the external defibrillator 10 may be turned on, the external defibrillator 20 may transmit the control command inputted by the user and/or the trigger command generated by the host to the expander 20, and both the external defibrillator 10 and the expander 20 may be powered on. The expander 20 can operates according to the control and/or trigger command received, for example, collecting vital sign data, printing report, and the like, thereby realizing various expanded functions that the external defibrillator cannot provide. Vital sign data collected by the host 22 of the expander 20 can be sent to the external defibrillator 10 through the second communication interface 24 and then displayed by the first human-computer interaction unit of the external defibrillator 10.

When the external defibrillator 10 is needed to be taken to the rescue sites, the external defibrillator 10 can be removed from the expander 20 by opening the locking wrench 282 on the expander 20, while the expander 20 is still working. The external defibrillator 10 can then be taken to the rescue sites for defibrillation, and the ECG data and/or defibrillation data collected by the external defibrillator 10 onsite can be transmitted to the expander 20 via the first communication interface 12. When the external defibrillator 10 is brought back and affixed on the expander 20, vital sign data collected by the host 22 can be transmitted to the external defibrillator 10 for display. The ECG data and/or defibrillation data collected by the external defibrillator 10 and the vital sign data collected by the host 22 may be printed by the recorder 222.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, advantages, and solutions to problems have been described above with regard to various embodiments and are not to be construed as critical, required, or essential feature or element. The scope of the present disclosure should, therefore, be determined by the following claims.

What is claimed is:

1. An external defibrillator, comprising a first human-computer interaction unit, a main control unit and a power module, wherein the external defibrillator further comprises:
a first communication interface for transmitting data with an expander corresponding to said external defibrillator.

2. The external defibrillator of claim 1, wherein said data transmitted by the first communication interface comprises one or more of the following data: vital sign data outputted by said expander, control and trigger commands outputted by the main control unit.

3. The external defibrillator of claim 2, wherein said data transmitted by the first communication interface further comprises at least 1-lead ECG data and/or defibrillation data collected by the main control unit; and/or
said first human-computer interaction unit displays said vital sign data outputted by said expander.

4. The external defibrillator of claim 1, wherein said first communication interface comprises a first cable communication interface and/or a first wireless communication interface.

5. The external defibrillator of claim 4, wherein the first wireless communication interface adopts one or more of the following communication protocols: infrared, Bluetooth, WiFi, Zigbee and RF.

6. The external defibrillator of claim 4, wherein said first wireless communication interface communicates with a remote terminal and/or a remote server.

7. The external defibrillator of claim 1, further comprising:
a charging interface for charging the power module.

8. The external defibrillator of claim 7, wherein said charging interface is a cable charging interface or a wireless charging interface.

9. The external defibrillator of claim 1, further comprising:
a first locking device for affixing the external defibrillator on the expander.

10. The external defibrillator of claim 9, wherein said first locking device comprises at least one first recess.

11. The external defibrillator of claim 1, wherein said external defibrillator is an auto external defibrillator (AED).

12. An external defibrillator expander, comprising:
a host for collecting vital sign data; and
a second communication interface, connected with the host, for transmitting data with an external defibrillator.

13. The expander of claim 12, wherein the data transmitted by the second communication interface comprises one or more of the following data: vital sign data outputted by said expander and control and trigger commands outputted by said external defibrillator.

14. The expander of claim 13, wherein the data transmitted by the second communication interface further comprises ECG data and/or defibrillation data;
said host further comprises:
a recorder for printing said ECG data and defibrillation data received by said second communication interface and one or more vital sign data collected by said host.

15. The expander of claim 14, wherein said recorder has a specification of 50 mm, 80 mm, 114 mm or 210 mm.

16. The expander of claim 12, wherein said second communication interface comprises a second cable communication interface and/or a second wireless communication interface.

17. The expander of claim 16, wherein the second wireless communication interface adopts one or more of the following communication protocols: infrared, Bluetooth, WiFi, Zigbee and RF.

18. The expander of claim 12, further comprising:
a charging unit, corresponding to a charging interface of said external defibrillator, for charging said external defibrillator;
a second locking device for affixing said external defibrillator on said expander; and/or
a second human-computer interaction unit having an input device and a display device.

19. The expander of claim 18, wherein said second locking device comprises a locking wrench and at least one second recess corresponding to a first recess on said external defibrillator.

20. An defibrillation monitoring system, comprising an external defibrillator and an expander corresponding to said external defibrillator;
wherein said external defibrillator comprises a first human-computer interaction unit, a main control unit, a power module and a first communication interface for transmitting data with said expander;
wherein said expander comprises:
a host for collecting vital sign data; and
a second communication interface, connected with the host, for transmitting data with said external defibrillator.
